(19) United States
(12) Patent Application Publication
(10) Pub. No.: US 2012/0121578 A1
(43) Pub. Date: May 17, 2012

(54) METHODS OF USING IMMUNOGLOBULIN AGGREGATES

(75) Inventor: David Block, Baltimore, MD (US)

(73) Assignee: GLIKNIK INC., Baltimore, MD (US)

(21) Appl. No.: 13/320,183

(22) PCT Filed: May 13, 2010
(86) PCT No.: PCT/US10/34765

§ 371 Date: Jan. 27, 2012

Related U.S. Application Data
(60) Provisional application No. 61/177,860, filed on May 13, 2009.

(51) Int. Cl. A61K 39/395; A61P 37/06; A61P 25/00; A61P 1/04; A61P 29/00; A61P 3/10; A61P 31/04; A61P 7/00; A61P 37/04; A61P 35/00

(52) U.S. Cl. 424/130.1

(57) ABSTRACT

The current invention provides a method of treating a mammal in need of such treatment with aggregated IgG derived from pooled human plasma. The invention further provides using the discarded fraction obtained during the course of standard IgG fractionation as the source of the aggregated IgG. The methods of the invention further provide for enhancing the aggregation of the aggregate fraction as well as enriching and modifying glycoforms.

This application claims priority to U.S. Application No. 61/177,860 filed on May 13, 2009, the contents of which are hereby incorporated by reference in their entirety.

### BACKGROUND

Most commercially available immunoglobulins produced at high concentrations are derived from pooled human serum, collected, processed, and distributed for sale by the blood products industry. The first purified human immunoglobulin G (IgG) preparation used clinically was immune serum globulin which was produced in the 1940s (Cohn, E. J., et al “J. Am Chem. Soc., 68:459-475 (1946)) and Oncely, J. L. et al., J. Am Chem Soc. 71:541-550 (1949). The gammaglobulin produced by this method shows a molecular distribution having a high molecular weight, when analyzed by way of high resolution size exclusion chromatography.

Manufacturers' experiences with Intravenous Immunoglobulin (“IVIG”) have shown that these preparations are unstable in relatively dilute solutions, and the instability is manifested by the formation of insoluble particles by a process known as ‘shedding’ when the material is stored at room temperature (Fernandes and Lundband, Vox. Sang. 39:101-112 (1980)). In addition to shedding, concentrated solutions of IVIG have a tendency to aggregate during long term storage. As much as 10-30% (w/w) of the IVIG solution could be comprised of aggregates (Gronski et al. Behring Inst. Mitt. 82:124-143 (1988)).

Problems concerning the therapeutic use of gammaglobulin by intravenous infusion go back to the first preparations obtained by the Cohn-Oncley method, including its many variants, which caused the appearance of adverse reactions with a very high incidence, especially in agammaglobulinaemic patients receiving it (up to 90% of patients). The reactions described were associated with a reduction of the complement of patients treated by this route. (Barandun, et al. Vox. Sang, 7:157-174 (1962)).

The scientific and patent literature teaches the term “dimer” and “dimeric” to refer to the aggregation of more than one auto-dimerized immunoglobulin homodimer, which dimeric structure is generally referred to in the scientific and patent literature as a “monomer.”

It was observed that the gammagolobulin obtained by alcoholic fractionation has a notable capacity for spontaneously fixing complement as a result of the denaturing of the protein produced during the alcoholic fractionation process. In particular, the generation of high molecular weight gammaglobulin aggregates arising during the alcoholic fractionation process included antibody-antigen complexes having the capacity to freely fix complement. The majority of these aggregates are comprised of dimers produced by complexes of idiotypic and anti-idiotypic antibodies. Since monoclonal antibodies prepared from tissue culture supernatants do not contain anti-idotype antibodies, these idiotypic and anti-idiotypic antibody complexes are largely absent in a typical monoclonal antibody preparation. However, dimer and aggregate formation in these preparations can be caused by complexation between partially denatured monomeric antibody molecules. Mechanical stress such as that encountered during tangential flow ultrafiltration used for concentrating antibodies can also lead to an increase in aggregation. (Wang and Hanson, J. Parenteral Sci. Tehchnol. 42:S3-S26 (1988)).

Most of the world's intravenous immunoglobulin (IVIG) is produced in accordance with the 1940s method of Cohn and Oncley. The sequential precipitation of proteins by increasing the concentrations of ethanol was designed to result in a stable solution of human albumin. Fraction II of Cohn's original method is precipitated at 25% ethanol and is 95-98% pure IgG. The Kistler-Nitshmann modification of Cohn's scheme simplified the method somewhat to allow access to the two main fractions with fewer steps, while retaining the basic ethanol precipitation chemistry. (Kistler and Nitschman, Vox Sang. 7:414-424 (1962)).

In standard preparations, as much as 20% of the IgG content can be in the form of dimers of the immunoglobulin homodimers and larger aggregates (Young et al., J. Biol. Stand. 6:27-43 (1978)). Modification of the manufacturing method specifically designed to eliminate aggregates as an improvement to the final IVIG product has resulted in the development of three product generations of IVIG.

The first precipitations were treated with proteolytic enzymes to cleave aggregates (Sgouris, Vox. Sang, 13:71-84 (1967)). This led to preparations that were well tolerated intravenously but were rapidly catabolized in vivo and are now obsolete (Boshkov, & Kelton, Transfusion Med. Rev. 3:82-120 (1989)).

Second generation-products were treated to modify the IgG chemically so as to eliminate aggregates. These treatments included beta-propiolactone, S-sulphonation and reduction and alkylation. (U.S. Pat. No. 3,916,026, Masuho et al, Vox. Sang, 32:175-181 (1977), and Ferndandes and Lundblad, Vox. Sang., 39:101-112 (1980)).

Most of the third generation manufacturing processes achieve the desired removal of IgG aggregates while retaining IgG in a native form. These include removal of aggregates by polyethylene glycol precipitation and low pH incubation in the presence of small amounts of enzymes. Once removed from the IVIG preparation these aggregates are currently discarded as undesirable waste product.

Historically, it has been believed that preparations substantially free of these aggregates were the most useful and safe in IVIG preparations. However it was discovered that in fact the aggregate portion of the resulting IVIG was actually the more useful fraction. Three lines of evidence suggest that additional stimulation of these inhibitory receptors by these naturally occurring immune aggregates contained within the IVIG preparation are either required and/or facilitate the anti-inflammatory effects of IVIG.

First, in clinical studies of ITP, the anti-inflammatory effects of IVIG directly correlate with the presence of immune aggregates in the sera (Augener et al. Blut., 50:249-252 (1985).). Furthermore, IM administration of anti-D immunoglobulin, containing a high aggregate percentage, improves platelet counts in Rh negative patients (Bussel Blood, 77:1884-1893 (1991). These data provide a clear temporal association between the presence of immune aggregates and the anti-inflammatory effects of IVIG in the clinical setting.

Second, several reports clearly demonstrate that aggregated IVIG products are more effective than non-aggregated forms in ameliorating inflammation in murine models. For example, Teeling et al. demonstrated that aged IVIG, containing a high percentage of dimers of Fc dimers, effectively treated a murine model of ITP lasting for several days, while monomeric IVIG was not therapeutic (Teeling Blood, 98:1095-1099 (2001)). Similarly, in an elegant model, using a mouse anti-human Fc monoclonal antibody (“mAb”) to create tetramolecular complexes, Bazin and colleagues demonstrated that these artificial immune complexes were much more potent than high dose IVIG in protecting animals against ITP (Bazin, et al. British J. of Haematology. 2004;127:90-96 (2004)). These data clearly demonstrate that natural or artificially induced aggregates of human immunoglobulins in IVIG are more efficient than non-aggregated constructs in protecting against ITP.

Third, mAbs which are aggregated by specific antibody-antigen interactions with soluble proteins can effectively treat murine models of ITP and arthritis, while the un-aggregated forms are ineffective (Siragam, et al., J Clin Invest. 115:155-160 (2005)). Specifically, Siragam and colleagues have clearly demonstrated that both OVA-anti-OVA conjugates and anti-OVA-(RBC-OVA) conjugates, but not the individual components of these products are therapeutically efficacious.

Miesscher and Bolli describe a pharmaceutical formulation using the so-called dimer fraction prepared from IVIG, i.e., aggregate and multimers of the immunoglobulin homodimers, for use in the treatment of many disorders. (U.S. Pat. Publ. No.: 2009/0028846, incorporated by reference herein in its entirety). Specifically, Misscher and Bolli teach that size fractionated dimeric IgG give rise to antibodies with increased reactivity to antigens and with higher affinity to target antigens that may have a higher biological activity.

The dimeric composition of Misscher and Bolli is derived from the purified IVIG product and separated from the monomeric fraction (i.e. immunoglobulin homodimers). Specifically, the dimeric fraction is obtained by size fractionation of human IVIG preparations and is characterized by an increased reactivity to exoantigens such as tetanus toxoid and/or respiratory syncytial virus (RSV) and/or the Toxin A of Pseudomonas aeruginosa and/or autoantigens such as red blood cell ghosts and/or Hep-2 epithelial cells. This process, however is both time consuming, and expensive.

Protein glycosylation also plays an important role in many important biological recognition processes such as protein folding and quality control, cell adhesion, host-pathogen interaction, development, and immune responses. (Varki, Glycobiol. 3:97-130 (1993); Dwek, et al. Nat. Rev. Drug Disc. 1:65-75 (2002); Helenius and Aebi, Science 291:2364-2369 (2001); Dube and Bertozzi, Nat. Rev. Drug Disc., 4:477-488 (2005); Haltiwanger, Annu. Rev. Biochem. 73:491-537 (2001); Dwek, Chem. Rev. 96:683-720 (1996)). Specifically, human antibodies of the IgG class are glycoproteins that carry a conserved N-glycan at the Asn-297 of the Fc domain. Furthermore, recent studies have suggested that by varying the attached N-glycan one can differentially impact the effector functions of monoclonal antibodies and confer upon them diverse therapeutic properties.

Therefore, a need exists to more efficiently and cost effectively isolate the active aggregate immunoglobulin fraction without going through essentially two rounds of purification. The current inventor has surprisingly found, contrary to what was previously thought, the aggregate fraction of unfractionated or partially fractionated pooled serums is not only not harmful, but surprisingly beneficial. The inventor has further discovered that the discarded aggregate fraction of pooled human serum is more potent than conventional fractionated serum. Furthermore, the inventor has also surprisingly found that by enriching the aggregate IgG for preferred glycoforms, by subjecting the aggregate IgG to glycoslyation reactions, or by taking actions to further enrich the percentage and size of IgG aggregates, the effector function and potency of the aggregate IgG from unfractionated or partially fractionated pooled plasma is further enhanced.

Further examples and disclosures related to the general field can be found, for example, in U.S. Pat. No. 6,252,055 B1, U.S. Published Application No. 2002/0151688 A1 and PCT Publication No. WO 99/64462, all of which are incorporated by reference in their entireties.

### BRIEF DESCRIPTION OF THE DRAWINGS

FIG. 1 shows one fractionation process which can be used to obtain the aggregates useful in the methods of the current invention.

### SUMMARY OF THE INVENTION

The present invention relates to methods of obtaining and then using from pooled human plasma immunoglobulin aggregate fractions normally discarded from the fractionation process currently used in manufacturing intravenous immunoglobulin (IVIG) compositions.

According to the present invention, it was found that it is not necessary to purify the IVIG preparation in order to separate the dimeric or aggregate fraction. Rather, the currently discarded fraction of dimeric, multimeric, and aggregated human IgG removed from pooled human plasma in the IVIG preparation process by polyethylene glycol precipitation or low pH or similar processes during the Cohn-Oncley process or similar processes contains the useful aggregation fraction. By utilizing this discarded fraction of the purification process, the inventors are able to maximize the yield of useful immunoglobulin concentrations while at the same time reducing the time and expense of going through the entire Cohn-Oncley process or similar processes only to isolate for use or to remove aggregates from the final IVIG product.

The present invention relates to a method of treating a mammal in need of such treatment comprising administering to said mammal a pharmaceutical composition comprising aggregate IgG derived from pooled plasma.

In one embodiment of the invention, the aggregate IgG is derived from precipitation of the IgG fraction during a Cohn-Oncley fractionation process. In a further embodiment, the IgG aggregates are obtained from polyethylene glycol precipitation of IgG fractions during the Cohn-Oncley fractionation process.

In another embodiment, the IgG aggregates are obtained from using low pH precipitation. In another embodiment, the IgG aggregates are obtained from using bother polyethylene glycol precipitation and low pH precipitation.

In one embodiment, the aggregate IgG comprises more than about 30% of the IgG in aggregate form as compared to monomeric form. In another embodiment, the aggregate IgG comprises more than about 50% of the IgG in aggregate form as compared to monomeric form. In still another embodiment, the aggregate IgG comprises more than about 90% of the IgG in aggregate form as compared to monomeric form.

In one embodiment of the current invention, the mammal in need of such treatment is a mouse, a rat, a rabbit, a non-human primate, a doge, a cat, a pig, a goat a sheep or a human. In one embodiment, the mammal is a human.

In one embodiment of the current invention, the mammal in need of such treatment is in need of such treatment for an autoimmune disease. In another embodiment, the mammal in need of such treatment is in need of such treatment for a neurological disorder. In still another embodiment, the mammal in need of such treatment is in need of such treatment for caner. In one embodiment, them mammal is need of such treatment for severe vasculitis, rheumatoid arthritis, Systemic Lupus Erythematosus, multiple sclerosis, graft vs. host disease, psoriasis, juvenile onset diabetes, Sjogren's' disease, thyroid disease, myasthenia gravis, transplant rejection, inflammatory bowel disease, immune thrombocytopenic purpura, Guillain Bane Syndrome, Kawasaki disease, chronic inflammatory demyelinating polyneuropathy, primary hypo/agammaglobulinemia, common variable immunodeficiency, Wiskott-Aldrich syndrome, primary immune deficiencies including severe combined immunodeficiency, secondary hypo/agammaglobulinemia including in patients with chronic leukemia and myeloma, AIDS, bacterial infections, allogenic bone marrow transplant, multifocal motoric neuropathy, Eaton-Lambert's syndrome, Opticus Neuritis, epilepsy, Abortus habitualis, primary antiphospholipid syndrome, scleroderma, vasculitis, Wegener's granulomatosis, autoimmune neutropenia, autoimmune hemolytic anemia, neutropenia, Crohn's disease, ulcerative colitis, celiac disease, septic shock syndrome, chronic fatigue syndrome, sinusitis, dilated cardiomyopathy, endocarditis, atherosclerosis and asthma.

In one embodiment of the current invention, the mammal is treated with said pharmaceutical composition comprising aggregate IgG derived from pooled human plasma at a dose of <1 gm/kg/day. In another embodiment, the mammal is treated at a dose of <0.5 gm/kg/day. In still another embodiment, the mammal is treated at a dose of <0.1 gm/kg/day.

In one embodiment of the current invention, the pharmaceutical composition is administered to the mammal in need thereof intravenously. In another embodiment, the composition is administered to the mammal intraperitoneally. In still another embodiment, the composition is administered to the mammal intra-arterially. In yet another embodiment, the composition is administered to the mammal sub-lingually. In still another embodiment, the composition is administered to the mammal via infusion into the cerebrospinal fluid.

In one embodiment of the present invention, the pharmaceutical composition is administered to the mammal once daily. In another embodiment, the pharmaceutical composition is administered twice daily. In yet another embodiment, the pharmaceutical composition is administered once weekly. In still another embodiment, the pharmaceutical composition is administered once monthly. In another embodiment, the pharmaceutical composition is administered in monthly divided doses over two to five days.

In one embodiment of the present invention, the aggregate IgG has been subject to a glycosylation modification or enrichment of a preferred glycoform. In a further embodiment, the glycosylation modification modifies an N-terminal asparagine residue in the Fc region of an immunoglobulin or antibody. In a further embodiment, the glycosylation modification increases IgG aggregate binding to immune cell surface receptors. In one embodiment, the immune cell surface receptor is an immunoglobulin superfamily member. In a further embodiment, the immunoglobulin superfamily member is an Fc gamma receptor. In another embodiment, the immune cell surface receptor is a C-type lectin receptor. In a further embodiment, the C-type lectin receptor is DC-SIGN.

In one embodiment, the pooled plasma is subjected to further aggregation prior to fractionation. In another embodiment, the aggregate IgG is subjected to further aggregation after fractionation. In yet another embodiment, the further aggregation is a result of administration of heat, administration of time delays, adsorption to microparticle surfaces, administration of chemicals to increase immunoglobulin aggregation, administration of buffer ions, or combinations thereof.

### DETAILED DESCRIPTION OF THE INVENTION

The present invention relates to a method of treating a mammal with the aggregate fraction of IgG derived from pooled human plasma, for instance, as a by-product of the Cohn-Oncley fractionation process. Specifically excluded from this invention is treating a mammal with the aggregate fraction of IgG derived from purified human Intravenous Immunoglobulin.

By the term “mammal” is meant any warm-blooded animal, preferably the mammal is a mouse, rat, rabbit, non-human primate, dog, cat, pig, goat, sheep, or human. Most preferably, the mammal is a human.

The following definitions may be used to understand the invention, but are meant to encompass scientific equivalents.

As used in this application, the singular form “a,” “an,” and “the” include plural references unless the context clearly dictates otherwise. For example, the term “an agent” includes a plurality of agents, including mixtures thereof.

As used herein, the terms “Immunoglobulin,” “antibody,” and “antibodies” refer to monoclonal antibodies, multispecific antibodies, human antibodies, humanized antibodies, synthetic antibodies, chimeric antibodies, polyclonal antibodies, single chain antibodies, Fab fragments, F(ab′) fragments, disulfide-linked bispecific Fvs (sdFv), intrabodies, and anti-idiotypic (anti-Id) antibodies (including, for example, anti-Id and anti-anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

By the term “aggregate fraction,” is meant a fraction of immunoglobulins obtained from fractions 2 and 3 of the Cohn-Oncley fractionation process of pooled human plasma wherein a fraction of immunoglobulins are not in monomeric form. More specifically, the “aggregate fraction” will contain dimers and higher order aggregates of immunoglobulins. In a preferred embodiment the “aggregate fraction” will contain greater than about 20% aggregate IgG when compared to the amount of monomeric IgG in the fraction. In a more preferred embodiment the “aggregate fraction” will contain more than 40% aggregate IgG when compared to the amount of monomeric IgG. In an even more preferred embodiment the “aggregate fraction” will contain more than about 50% aggregate IgG when compared the amount of monomeric IgG. In the most preferred embodiment the “aggregate fraction” will contain more than about 90% aggregate IgG when compared to the amount of monomeric IgG. In other embodiments, the aggregate fraction contains more than about 10, 20, 30, 40, 50, 60, 70, 75, 80, 85, 90, 95 or 99% aggregate IgG.

By the term “by-product of the Cohn-Oncley fractionation process” is meant, the aggregate portion derived from precipitation of the IgG fraction of Cohn-Oncley by polyethylene glycol precipitation, pH precipitation, or any other precipitation method known in the art used to remove aggregate IgG from the fractionation process, and all combinations thereof. A person of ordinary skill in the art will recognize that modifications may be made to the Cohn-Oncley process and to the immunoglobulin multimer and aggregate precipitation process without substantially altering the concept of isolating immunoglobulin aggregates during the process of manufacturing IVIG and using those immunoglobulin aggregates as described herein.

Aggregate immunoglobulins derived from pooled human plasma can be further aggregated by any known process in the art. For example, aggregation may be enhanced by sterile filtering the fractionated IgG and placing the resulting fraction in a 37° C. incubator for two weeks. Additionally, immunoglobulins may be further aggregated by the administration of heat or time delays, or adsorption onto microparticle surfaces (i.e. tungsten, silica, iron, alumina or titania) or administration of chemicals (i.e. chondroitin sulphates) or the administration of buffer ions. The further aggregation can take place prior to fractionation or after fraction of pooled serum.

Aggregate immunoglobulins derived from pooled human plasma can enriched for preferred glycoforms. For example, defucosylated immunoglobulin and 2,6 sialylated immunoglobulin are known to enhance binding to Fc gamma receptors and to DC-SIGN/SIGN-R1. The enrichment of these, as well as other, glycoforms can be done by methods well known in the art. For example, through hydrazide chemistry or lectin affinity chromatography. The enrichment of preferred glycoforms can be done at any time during the fractionation process. For example, the enrichment step can be done prior to fractionation, after fractionation in the aggregate fraction, after fractionation on the non-aggregate fraction. If the enrichment step is done after fractionation, the enrichment can be done prior to or after further aggregation of aggregate containing fraction or the non-aggregate fraction.

Alternatively, aggregate immunoglobulins from pooled human plasma can be subjected to glycosylation modifications. Aggregate IgG can be deglycosylated, for example by PNGase F, or by the activity of endo-β-N-acetylglucosaminidases (EnGases). A non-limiting list of EnGases include, Endo-A from Arthrobactor protophormiae which is specific for high-mannose type N-glycans, and Endo M from Mucro hiemalis that can act on both high-mannose and complex type N-glycans. Following deglycosylation, the aggregate immunoglobulins can be reglycosylated to achieve the desired glycoform, for example by adding N-acetlyglucosamine, mannose, galactose, fructose or sialic acids. Aggregate immunoglobulins may be subject to glycosylation modification prior to or after fractionation, prior to or after further aggregation and/or prior to or after glycoform enrichment.

Aggregate immunoglobulins derived from pooled human plasma intended for therapeutic use will generally be administered to the patient in the form of a pharmaceutical formulation. Such formulations preferably include, in addition to the immunoglobulin, a physiologically acceptable carrier or diluent, possibly in admixture with one or more other agents such as other immunoglobulins or drugs, such as an antibiotic. Suitable carriers include, but are not limited to, physiologic saline, phosphate buffered saline, glucose and buffered saline, citrate buffered saline, citric acid/sodium citrate buffer, maleate buffer, for example malic acid/sodium hydroxide buffer, succinate buffer, for example succinic acid/sodium hydroxide buffer, acetate buffer, for example sodium acetate/acetic acid buffer or phosphate buffer, for example potassium dihydrogen orthophosphate/disodium hydrogen orthophosphate buffer. Optionally the formulation contains Polysorbate for stabilisation of the antibody. Alternatively the immunoglobulin may be lyophilised (freeze dried) and reconstituted for use when needed by the addition of water and/or an aqueous buffered solution as described above.

Therapeutic formulations of the aggregate immunoglobulin may be prepared for storage by mixing the aggregate immunoglobulin having the desired degree of purity with optional physiologically acceptable-carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the aggregate immunoglobulin, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Although there is variability in dosing of IVIG by disease, most human dosing is approximately 1-2 gram per kilogram of body weight which may be divided into two to five consecutive day doses and repeated every 1-2 months. According to a further aspect of the present invention, the aggregate immunoglobulin preparation derived from pooled human plasma is given at a dose of less than 1 gram per kilogram of body weight because the aggregate fraction is a more active component than the monomer fraction in treating human disease. However, dosages may range from about 0.01 to about 200 mg/kg of body weight or greater, for example from 0.01 to 30 mg/kg of body weight, for example about 0.1, about 1, about 10, about 20, about 30, about 50, or about 75 mg/kg of body weight, with 1 to 75 mg/kg being preferred and about20 to about 50 mg/kg being especially preferred.

Administration of the pharmaceutical composition comprising the aggregated immunoglobulin of the present invention, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances the aggregate immunoglobulin may be applied as a solution or spray to the affected area.

Administration of the pharmaceutical composition comprising aggregate immunolgobulin of the present invention may be done prior to, during or following the administration of an additional therapeutic agent(s). Non-limiting examples of additional therapies or therapeutic agents(s) useful in combination with aggregate immunoglobulin compositions of the present invention include, chemotherapy, radiation therapy, surgical resection, immunosuppressants, hormones, anti-histamines, anti-inflammatories, vitamins, sterioids or combinations thereof.

According to a further aspect of the invention the aggregate immunoglobulin preparation derived from pooled human plasma is envisaged for the use in human therapy. Various human disorders can be treated such as cancer, infectious diseases, autoimmune disorders, inflammatory diseases, or immune dysfunction. Specifically, disorders such as severe vasculitis, rheumatoid arthritis, Systemic Lupus Erythematosus, multiple sclerosis, graft vs. host disease, psoriasis, juvenile onset diabetes, Sjogren's' disease, thyroid disease, myasthenia gravis, transplant rejection, inflammatory bowel disease, immune thrombocytopenic purpura, Guillain Barre Syndrome, Kawasaki disease, chronic inflammatory demyelinating polyneuropathy, primary hypo/agammaglobulinemia, common variable immunodeficiency, Wiskott-Aldrich syndrome, primary immune deficiencies including severe combined immunodeficiency, secondary hypo/agammaglobulinemia including in patients with chronic leukemia and myeloma, AIDS, bacterial infections, allogenic bone marrow transplant, multifocal motoric neuropathy, Eaton-Lambert's syndrome, Opticus Neuritis, epilepsy, Abortus habitualis, primary antiphospholipid syndrome, scleroderma, vasculitis, Wegener's granulomatosis, autoimmune neutropenia, autoimmune hemolytic anemia, neutropenia, Crohn's disease, ulcerative colitis, celiac disease, septic shock syndrome, chronic fatigue syndrome, sinusitis, dilated cardiomyopathy, endocarditis, atherosclerosis and asthma can be treated with the aggregate IgG of the current invention. Also provided is a method of treating a human being having any such disorder comprising administering to said individual a therapeutically effective amount of a preparation according to the invention.

### EXAMPLES

### Example 1

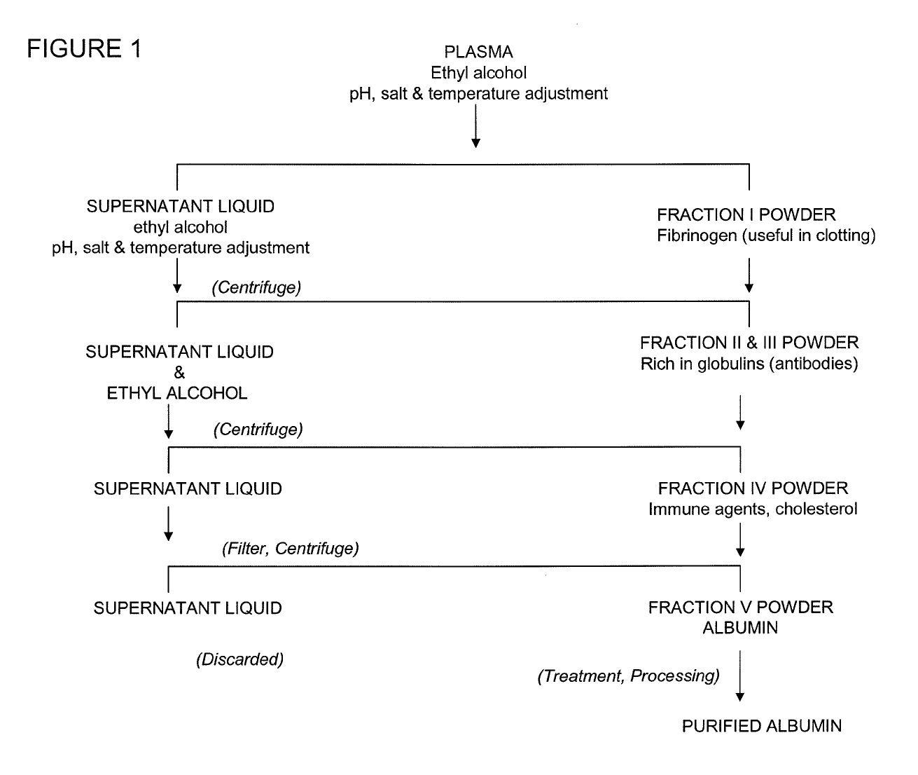

The fractionation process is carried out, for example, as depicted in FIG. 1 ((Cohn, E. J., et al “J. Am Chem. Soc., 68:459-475 (1946)) and Oncely, J. L. et al., J. Am Chem Soc. 71:541-550 (1949).). Specifically, plasma is collected and pooled from thousands of human donors and fractionated by subjecting the plasma to increasing concentrations of ethyl alcohol in sequential steps. First, fraction I is obtained by precipitating the plasma with ethyl alcohol. The powder obtained from this fractionation step contains fibrinogen, which is useful in clotting. The supernatant liquid is then fractionated again with ethyl alcohol. The resulting fractions II and III are rich in immunoglobulins. As discussed in Farrugia and Poulis, Transfusion Medicine, 11 :63-74 (2001), which is herein incorporated by reference in its entirety, as much as 20% of the IgG content is in the form of dimers and larger aggregates. It is from this step that aggregates are removed, for example with polyethylene glycol or incubation at low pH, or any means suitable for removing aggregates. Traditional methods would discard this aggregate portion as undesirable by-product of the material in the production process of IVIG; however, it is this aggregate that is the subject material of the present invention. The aggregate is removed and stored for use in pharmaceutical preparations and for the treatment of the diseases disclosed above. The supernatant obtained from this fractionation step can be further fractionated with ethyl alcohol to produce fraction IV which contains, immune agents and cholesterol. The supernatant is then centrifuged and filtered to obtain fraction V containing crude albumin which can then be treated and processed to produce purified albumin.

### Example 2

The murine preventative ITP model is a well-established and accepted model for assessing potential treatments for human patients with ITP. In this model, baseline platelet counts are measured in C57B/6 mice by serial tail vein nicking on day 1 of the experiment and then daily for 5 days. Ten microliters of blood is diluted in 15 μl citrate buffer. The samples are aspirated and analyzed for absolute platelet count on a cytometer. Control mice (the “ITP group”) will receive no pre-treatment and no platelet depletion. ITP group mice will receive only platelet depletion with MwReg30 anti-platelet antibody (BD Biosciences) administered by i.p. injection according to the manufacturer's instructions each afternoon on days 2-4. A single dose of active drug is given at Day 1 after the baseline platelet count. In this experiment, IVIG mice will receive Day 1 treatment with 2 gram/Kg body weight of human IVIG by IP injection, and platelet depletion with MwReg30 anti-platelet antibody administered by i.p. injection each afternoon on days 2-4. Three groups of mice receiving aggregate IgG derived from pooled human plasma will receive Day 1 treatment with 2 gram/Kg body weight IP, or 200 mg/kg body weight IV in one group; 1 gram/Kg body weight IP or 100 mg/Kg body weight IV, in a second group; and 500 mg/Kg body weight IP or 50 mg/Kg body weight IV of human aggregate IgG by IV injection and platelet depletion with MwReg30 anti-platelet antibody will be administered by IP injection each afternoon on days 2-4. The mice receiving aggregate IgG will have platelet counts in excess of the ITP group and comparable or better in comparison with the IVIG-treated group.

### Example 3

The therapeutic murine Collagen-Induced Arthritis (“CIA”) model is a well established and predictive model for the efficacy of therapeutic compounds in rheumatoid arthritis. IVIG is effective in this model. This model is well suited to assess aggregated IgG from pooled plasma as a therapeutic drug. In this model, collagen is injected and thereby arthritis is induced in the mouse. Drugs can be assessed for the ability to ameliorate or reverse worsening arthritis. At day 0 DBAI/J mice will be injected with bovine Type II collagen solution in a 1:1 mixture with Complete Freund's Adjuvant. At day 20 the collagen-immunization will be repeated except for one control group of 10 animals that will receive no collagen and are expected not to develop arthritis. From day 22-27 the mice that will have received collagen injections will be scored every other day for development of arthritis. Each paw will receive a score as follows: 0=no visible effects of arthritis; 1=edema and/or erythema of 1 digit; 2=edema and/or erythema of 2 digits; 3=edema and/or erythema of more than 2 digits; 4=severe arthritis of entire paw and digits. A calculated Arthritic Index (AI) score will be obtained by addition of individual paw scores and recorded at each measurement with a maximum AI=16. Mice will be selected into groups for treatment when they have an AI score of 3 and grouped for treatment. On the day when a group with an AI=3 is formed, testing with compound will begin in that group. A second control group will receive no therapeutic treatment. In this experiment, groups will be individually treated with: a) IVIG 2 gm/Kg administered IP every day as the protein load is too great to allow IV injection, b) aggregated IgG from pooled plasma 200 mg/Kg IV every 4 days (“q4D”), c) aggregated IgG from pooled plasma 100 mg/Kg IV q4D, d) aggregated IgG from pooled plasma 50 mg/Kg IV q4D, and e) aggregated IgG from pooled plasma 20 mg/Kg IV q4D. Each group will be treated for 4 doses at treatment days 0, 4, 8, and 12 and the mice observed through day 21. The mice receiving aggregated IgG from pooled plasma will have AI scores significantly lower than the no treatment control group and comparable or better in comparison with the IVIG-treated group.

### Example 4

The same murine preventative ITP and therapeutic CIA models described in examples 2 and 3 are well suited to assess aggregated IgG that has been modified with glycosylation changes, enriched for preferred glycoforms, or been modified by techniques that increase IgG aggregation. Aggregated IgG will be enriched for glycoforms that are known to modulate immunoglobulin binding to Fc gamma receptors and to DC-SIGN/SIGN-R1 including defucosylated immunoglobulin and 2,6 sialylated immunoglobulin. Enrichment of preferred glycoforms of aggregated IgG will be produced using, for example, affinity chromatography columns with different immobilized lectins. Alternatively or in addition IgG aggregation will be enhanced by sterile filtering the IgG aggregate and placing in a 37 C incubator for two weeks

Mice receiving aggregate IgG derived from pooled human plasma enriched for preferred glycoforms, with modified glycosylation, and/or enriched for IgG aggregates will be studied in the ITP and CIA experiments as outlined in Examples 2 and 3.

All publications, patents and patent applications herein are incorporated by reference to the same extent as if each individual publication or patent application was specifically and individually indicated to be incorporated by reference.

What is claimed:

1. A method of treating a mammal in need of such treatment comprising administering to said mammal a pharmaceutical composition comprising aggregate IgG derived from pooled plasma.

2. The method of claim 1, wherein said aggregate IgG is derived from precipitation of the IgG fraction during a Cohn-Oncley fractionation process, polyethylene glycol precipitation, and/or low pH precipitation.

3-5. (canceled)

6. The method of claim 1, wherein said aggregate IgG comprises more than about 20-90% of the IgG in aggregate form as compared to monomeric form.

7-9. (canceled)

10. The method of claim 1 wherein said mammal is a mammal selected from the group consisting of mouse, rat, rabbit, non-human primate, dog, cat, pig, goat, sheep, or human.

11. (canceled)

12. The method of claim 1 wherein said mammal in need of such treatment is in need of such treatment for an immunodeficiency disorder, an autoimmune disorder, a neurological disorder, or cancer.

13-15. (canceled)

16. The method of claim 1 wherein said mammal in need of such treatment is in need of such treatment for a condition selected from the group consisting of severe vasculitis, rheumatoid arthritis, Systemic Lupus Erythematosus, multiple sclerosis, graft vs. host disease, psoriasis, juvenile onset diabetes, Sjogren's' disease, thyroid disease, myasthenia gravis, transplant rejection, inflammatory bowel disease, immune thrombocytopenic purpura, Guillain Barre Syndrome, Kawasaki disease, chronic inflammatory demyelinating polyneuropathy, primary hypo/agammaglobulinemia, common variable immunodeficiency, Wiskott-Aldrich syndrome, primary immune deficiencies including severe combined immunodeficiency, secondary hypo/agammaglobulinemia including in patients with chronic leukemia and myeloma, AIDS, bacterial infections, allogenic bone marrow transplant, multifocal motoric neuropathy, Eaton-Lambert's syndrome, Opticus Neuritis, epilepsy, Abortus habitualis, primary antiphospholipid syndrome, scleroderma, vasculitis, Wegener's granulomatosis, autoimmune neutropenia, autoimmune hemolytic anemia, neutropenia, Crohn's disease, ulcerative colitis, celiac disease, septic shock syndrome, chronic fatigue syndrome, sinusitis, dilated cardiomyopathy, endocarditis, atherosclerosis and asthma.

17. The method of claim 1, wherein said mammal is treated with said pharmaceutical composition comprising aggregate IgG derived from pooled plasma at a dose of <0.1-1 gm/Kg/day.

18. (canceled)

19. (canceled)

20. The method of claim 1, wherein said pharmaceutical composition is administered to said mammal intravenously, subcutaneously, intraperitoneally, intra-arterially, sub-lingually, or via infusion into the cerebral spinal fluid.

21-25. (canceled)

26. The method of claim 17, wherein said dose of said pharmaceutical composition is given once daily, twice daily, once weekly, or once monthly.

27-30. (canceled)

31. The method of claim 1, wherein said aggregate IgG is enriched for a specific glycoform.

32. The method of claim 31, wherein the specific glycoform is 2,3-sialated IgG, 2,6-sialated IgG, or defucosylated IgG.

33. (canceled)

34. (canceled)

35. The method of claim 31, wherein the aggregate IgG is enriched for a specific glycoform by lectin affinity chromatograhy.

36. The method of claim 1, wherein said aggregate IgG is subjected to a glycosylation modification.

37. The method of claim 36, wherein said glycosylation modification is a modification to an N-terminal asparagine residue in the Fc region of an immunoglobulin or antibody.

38. The method of claim 36, wherein said glycosylation modification increases IgG aggregate binding to immune cell surface receptors.

39. The method of claim 38, wherein said immune cell surface receptor is an immunoglobulin superfamily member or a C-type lectin receptor.

40. The method of claim 39, wherein said immunoglobulin superfamily member is an Fc gamma receptor.

41. (canceled)

42. The method of claim 39 wherein said C-type lectin receptor is DC-SIGN.

43. The method of claim 1, wherein said pooled plasma is subjected to further aggregation prior to fractionation or after fractionation.

44. (canceled)

45. The method of claim 43, wherein said further aggregation is a result of administration of heat, administration of time delays, adsorption to microparticle surfaces, administration of chemicals to increase immunoglobulin aggregation, administration of buffer ions, or combinations thereof.
